(19) United States
(12) Patent Application Publication
(10) Pub. No.: US 2009/0176674 A1
(43) Pub. Date: Jul. 9, 2009

(54) Personal care compositions containing cationic synthetic copolymer and a detersive surfactant

(75) Inventors: Marjorie Mossman Peffly, Cincinnati, OH (US); Kendrick Jon Hughes, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/338,592

(22) Filed: Dec. 18, 2008

Related U.S. Application Data
(63) Continuation-in-part of application No. 11/650,921, filed on Jan. 8, 2007.
(60) Provisional application No. 60/774,533, filed on Feb. 17, 2006.
(60) Provisional application No. 60/757,343, filed on Jan. 9, 2006.

(51) Int. Cl. A61K 8/81; A61K 8/73; A61Q 5/02

(52) U.S. Cl. 510/121; 510/122; 510/123; 510/125

(57) ABSTRACT

A personal care composition comprising:
a) a synthetic random copolymer having a net positive charge comprising;
   i.) a nonionic monomer unit of the following formula:

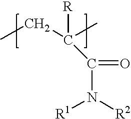

where R is H or C₁₋₄ alkyl; and R¹ and R² are independently selected from the group consisting of H, C₁₋₄ alkyl, CH₂OCH₃, CH₂OCH₂CH(CH₃)₂, and phenyl, or together are C₃₋₆cycloalkyl; and
ii.) a cationic monomer unit with 2 or more positive charges of the following formula:

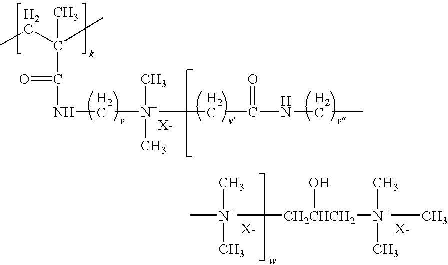

where k=1, each of v, v′, and v″ is independently an integer of from 1 to 6, w is zero or an integer of from 1 to 10, and X⁻ is an anion.
and;
b) an effective amount of a pyrithione or a polyvalent metal salt of a pyrithione;
c) an effective amount of a zinc-containing layered material;
d) a detersive surfactant; and
e) an aqueous carrier.

### CROSS REFERENCE TO RELATED APPLICATION(S)

This application is a continuation-in-part of U.S. application Ser. No. 11/650,921, filed Jan. 8, 2007, which itself claims the benefit of U.S. Provisional Application No. 60/774,533, filed Feb. 17, 2006; and U.S. Provisional Application No. 60/757,343, filed Jan. 9, 2006.

### FIELD OF THE INVENTION

The present invention relates to personal care compositions with improved lather and conditioning performance which comprise select synthetic copolymers.

### BACKGROUND OF THE INVENTION

Conditioning personal care compositions comprising various combinations of detersive surfactant and hair conditioning agents are known. These personal care compositions typically comprise an anionic detersive surfactant in combination with a conditioning agent such as silicone, hydrocarbon oil, fatty esters, or combinations thereof. These personal care compositions have become more popular among consumers as a means of conveniently obtaining hair conditioning and hair cleansing performance all from a single hair care product.

Many conditioning personal care compositions, however, do not provide sufficient deposition of conditioning agents onto hair or skin during the application process; if deposition is possible, it is only possible in formulations with relatively low levels of anionic surfactant. Without such deposition, large proportions of conditioning agent are rinsed away during the application process and therefore provide little or no conditioning benefit. Without sufficient deposition of the conditioning agent on the hair or skin, relatively high levels of conditioning agents may be needed in the personal care composition to provide adequate hair or skin conditioning performance. Such high levels of a conditioning agent, however, can increase raw material costs, reduce lathering, and present product stability concerns. Additionally, limitations on total anionic surfactant in order to form coacervate can limit the lather potential for a formula, or result in the need for higher levels of more expensive amphoteric surfactants to achieve good lather.

One known method for improving deposition of a hair conditioning agent onto hair involves the use of certain cationic deposition polymers. These polymers may be synthetic, but are most commonly natural cellulosic or guar polymers that have been modified with cationic substituents.

The formation of coacervate upon dilution of the personal care composition with water is important to improving deposition of various conditioning actives, especially those that have small droplet sizes (i.e., ≦2 microns). Dilution generally occurs during the usage of the personal care composition, for example, when a person applies a personal care composition to wet hair, the product is automatically diluted with water. In order to form coacervate, a personal care composition containing typical cationic polymers, such as natural cellulosic or guar polymers that have been modified with cationic substituents, tend to be significantly limited in total anion concentrations in order to achieve adequate levels of coacervate upon dilution. For example, limiting the total level of sulfate in a sulfated anionic surfactant will encourage coacervate formation but will limit the volume of lather that can be achieved with a particular personal care cleansing composition. Thus, for low cost, high lathering, coacervate forming compositions, it is desirable to use a cationic polymer that can form coacervate with higher levels of anionic surfactants.

A need still exists for improved conditioning and lather performance in personal care compositions.

It has now been found that select synthetic cationic polymers provide improved conditioning performance, especially wet hair conditioning, and improved deposition of dispersed hair conditioning agents onto hair or skin. These select polymers are especially effective at improving deposition of dispersed hair conditioning agents onto hair and skin, through coacervate formation upon dilution. In one embodiment, coacervate formation is optimized when formulated in combination with certain levels of anionic detersive surfactant in a personal care composition.

### SUMMARY OF THE INVENTION

The present invention meets the aforementioned need by providing a personal care composition comprising:
a) a synthetic random copolymer having a net positive charge comprising, based on the total number of monomeric units of the copolymer;
   i.) a nonionic monomer unit of the following formula:

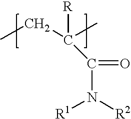

where R is H or C₁₋₄ alkyl; and R¹ and R² are independently selected from the group consisting of H, C₁₋₄ alkyl, CH₂OCH₃, CH₂OCH₂CH(CH₃)₂, and phenyl, or together are C₃₋₆cycloalkyl; and
   ii.) a cationic monomer unit with 2 or more positive charges of the following formula:

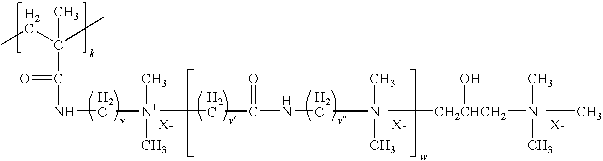

where k=1, each of v, v′, and v″ is independently an integer of from 1 to 6, w is zero or an integer of from 1 to 10, and X⁻ is an anion.
and;
b) an effective amount of a pyrithione or a polyvalent metal salt of a pyrithione;
c) an effective amount of a zinc-containing layered material;
d) a detersive surfactant; and
e) an aqueous carrier.

One embodiment comprises an anionic surfactant system having an optimized ethoxylate level and anion level.

These and other features, aspects, and advantages of the present invention will become evident to those skilled in the art from a reading of the present disclosure.

### DETAILED DESCRIPTION OF THE INVENTION

While the specification concludes with claims that particularly point out and distinctly claim the invention, it is believed the present invention will be better understood from the following description.

The personal care compositions of the present invention comprise a synthetic random copolymer, an effective amount of a pyrithione or a polyvalent metal salt of a pyrithione; an effective amount of a zinc-containing layered material; a detersive surfactant; and an aqueous carrier. Each of these essential components, as well as preferred or optional components, is described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “charge density” as used herein, means the ratio of the number of positive charges on a monomeric unit of which a polymer is comprised to the molecular weight of said monomeric unit. The charge density multiplied by the polymer molecular weight determines the number of positively charged sites on a given polymer chain.

The term “coacervate” as used herein, means the complex which forms between surfactant and polymer that may either be soluble or insoluble in the neat personal care composition, and which may become less soluble upon dilution and thus yielding an increase in its level of phase separation or precipitate in solution.

The term “comprising” means that unrecited steps, elements or other ingredients are not necessarily excluded. This term encompasses the terms “consisting of” and “consisting essentially of.” The compositions and methods/processes can comprise, consist of, and consist essentially of the essential elements and limitations of the invention described herein, as well as any of the additional or optional ingredients, components, steps, or limitations described herein.

The term “floc” as used herein, means localized clusters of agglomerated, insoluble coacervate. Any floc size disclosed herein is obtained using the HORIBA LA-910 Laser Diffraction Particle Size Analyzer and is reported as a volume average floc diameter.

The term “isotropic” as used herein, means a particular phase structure of coacervate wherein the structure is “identical along any three orthogonal directions in space, and is therefore dark or ‘nonbirefringent’ when viewed between crossed polarized light. (One direction is ‘orthogonal’ to another if the vector component of the first, in the direction of the second, is zero.) (Laughlin, R. G. (1994). “The Aqueous Phase Behavior of Surfactants,” 182, 8.2).

The term “linear charge density” as used herein, means the ratio of the number of positive charges on a monomeric unit of which the polymer is comprised to the length in Angstroms of said monomeric unit. The length of the monomeric unit is calculated by multiplying the ratio of the nonionic monomer by the length, in Angstroms, of the nonionic monomer plus the ratio of cationic monomer multiplied by the length, in Angstroms, of the cationic monomer.

The term “mass charge density” as used herein, means the ratio of the number of positive charges on a monomeric unit of which a polymer is comprised to the molecular weight of said monomeric unit. A molecular weight of the monomeric unit is calculated by multiplying the ratio of the nonionic monomer by the molecular weight of the nonionic monomer plus the product of the ratio of cationic monomer multiplied by the molecular weight of the cationic monomer.

The term “neat” as used herein, means the unadulterated form of the personal care composition (i.e., the altering of the present composition through dilution with water).

The term “polymer” as used herein shall comprise materials whether made by polymerization of one type of monomer or made by two (i.e., copolymers) or more (i.e., terpolymers) types of monomers.

The term “water insoluble” as used herein, means that the polymer is not soluble in water in the personal care composition. Thus, the polymer is not miscible with water. In general, solubility is determined at about 25° C.

The term “water soluble” as used herein, means that the polymer is soluble in water in the personal care composition. In general, the polymer should be soluble at about 25° C. at a concentration of at least about 0.1% by weight of the water solvent, preferably at least about 1%, more preferably at least about 5%, most preferably at least about 15%.

One embodiment is directed to the surprising discovery that compositions combining certain specific levels and ratios of surfactant as described by the overall anion and ethoxylate values maximize the conditioning benefit via maximization of coacervate formation. Moreover, while the optimum surfactant blend is different for each polymer, it has been discovered that the optimum surfactant composition can be described by two parameters. These parameters include anion and ethoxylate values that, when expressed as a function of the polymer's charge density and molecular weight, maximize the formation of coacervate.

Coacervates, without being limited to a particular theory, provide improved hair and skin conditioning without any additional conditioning actives. Further, when dispersed conditioning agent droplets are added to the matrix, the coacervate provides an improved mechanism for conditioning agent deposition, yielding conditioning agent deposition that results in even more of a conditioning benefit.

Synthetic Copolymer

The personal care compositions comprise synthetic copolymers that, in combination with the detersive surfactant component, an aqueous carrier and other optional components herein, form coacervate upon dilution. The polymers are formulated in a personal care composition that provides improved conditioning performance when formulated, even without additional conditioning actives, and also provides improved deposition of the conditioning agent onto the hair or skin. The monomer units of the synthetic copolymer may be arranged to form random copolymers and grafted copolymers. Random copolymers are preferred.

The concentration of the synthetic copolymer in the shampoo composition ranges about 0.01% to about 5%, preferably from about 0.05% to about 3%, more preferably from about 0.075% to about 1%, by weight of the composition.

Another embodiment comprises personal care compositions comprising a synthetic copolymer of sufficiently high molecular weight to effectively enhance the deposition of the conditioning active components of the personal care composition described herein. The average molecular weight of the synthetic copolymers is generally between about 10,000 and about 10 million, preferably between about 100,000 and about 3 million, still more preferably between about 200,000 and about 2 million.

In a further embodiment, the synthetic copolymers have mass charge densities of from about 0.1 meq/gm to about 6.0 meq/gm and more preferably from about 0.5 meq/gm to about 3.0 meq/gm, at the pH of intended use of the personal care composition. The pH will generally range from about pH 3 to about pH 9, and more preferably between about pH 4 and about pH 8.

In yet another embodiment, the synthetic copolymers have linear charge densities from at least about 2 meq/A to about 500 meq/A, and more preferably from about 20 meq/A to about 200 meq/A, and most preferably from about 25 meq/A to about 100 meq/A.

Nonionic Monomer Unit

The synthetic copolymers comprise the nonionic monomer unit represented by the following Formula I:

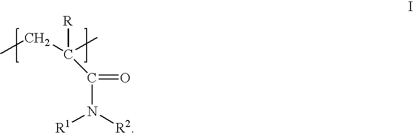

where R is H or C₁₋₄ alkyl; and R¹ and R² are independently selected from the group consisting of H, C₁₋₄ alkyl, CH₂OCH₃, CH₂OCH₂CH(CH₃)₂, and phenyl, or together are C₃₋₆cycloalkyl.

In one embodiment, nonionic monomer unit is acrylamide (AM), i.e., where R, R¹, and R² are all H as shown below:

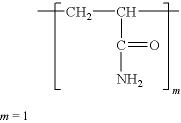

Another preferred nonionic monomer unit is methacrylamide (MethAM), i.e., where R is C₁ alkyl, and R¹ and R² are each H respectively:

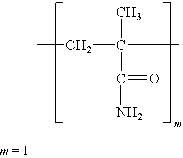

However, the other acrylamide derivatives within the scope of the formula set out above are also contemplated to be part of the present invention where polyacrylamide and copolymers using acrylamide monomers are useful.

The nonionic monomer portion of the synthetic copolymers is present in an amount from about 50% to about 99.5% by weight of the total copolymer. Preferably, this amount is from about 70% to about 99%, still more preferably from about 80% to about 99% by weight of the synthetic copolymer.

Cationic Monomer Unit

The synthetic copolymers also comprise the cationic monomer unit represented by Formula II:

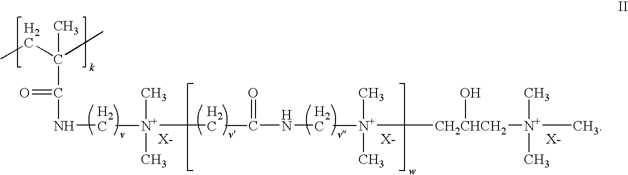

where k=1, each of v, v′, and v″ is independently an integer of from 1 to 6, w is zero or an integer of from 1 to 10, and X⁻ is an anion.

In one embodiment, a structure is present where k=1, v=3 and w=0, z=1 and X⁻ is Cl⁻ according to Formula II, above, to form the following structure:

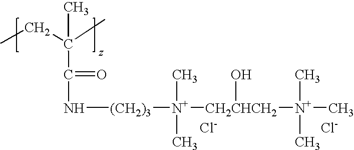

The above structure may be referred to as diquat.

Yet another embodiment is achieved by the structure formed wherein v and v″ are each 3, v′=1, w=1, y=1 and X⁻ is Cl⁻ according to Formula II, such as:

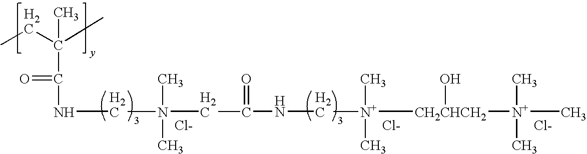

The above structure may be referred to as triquat.

Suitable cationic monomers can be made by, for example, the methods described in U.S. Patent Application Publication No. 2004/0010106 A1.

In one embodiment, the cationic monomer portion of the synthetic copolymers is present in an amount from about 0.5% to about 50% by weight of the total copolymer. Preferably, this amount is from about 1% to about 30% and most preferably from about 1% to about 20% by weight of the synthetic copolymer.

Method of Making the Triquat Monomer

Non limiting examples of polymerization techniques are described in U.S. Pat. No. 4,387,017, European Patent No. EP 156,646 and U.S. Patent Publication No. 2004/0010106 A1.

In one embodiment, the triquat monomer is formed by executing a three-step reaction in a jacketed reactor flask equipped with mechanical stirrer, gas inlet, condenser, and thermometer. The mechanical stirring and air purging is maintained throughout the reactions. First, 340.52 g of dimethylaminopropyl methacrylamide (DMAPMA), 238.75 g of methyl chloroacetate, 0.34 g of 4-methoxyphenol (MEHQ) and 425 g of methanol are added to the reactor and heated at about 65-70° C. for approximately 5 hours to yield (methacrylamidopropyl)(methoxy-carbonylmethyl)dimethylammonium chloride (MMDMAC). Samples are taken every 2 hours and analyzed by HPLC analysis and Cl titrated with AgNO₃ to ensure 100% conversion. Second, 0.365 g of MEHQ, and 224.5 g of dimethylaminopropylamine (DMAPA) is slowly added to MMDMAC solution after it is cooled to room temperature (about 25° C.). An exothermic reaction is observed, and the mixture appears light yellow in color. Heat is continued at about 65-70° C. for about 2 hours, then methanol is distilled out under vacuum. After confirming that all ester is converted into amide by HPLC in the second step, 637 g of 65% (3-chloro-2-hydroxypropyl)trimethylammonium chloride (Quat-188) is added. Third, the temperature is maintained at about 65-70° C. for about 2 hours. The reaction is continued in water for another hour to yield the triquat monomer. The triquat so synthesized is expected to contain a small amount of multiple quats as an impurity due to the slight excess use of chloroacetate and DMAPA. The multiple quats are not a concern for polymerization and for the uses of the triquat. If a highly pure triquat or multiple quats is required, the excess amount of chloroacetate and DMAPA can be removed under vacuum.

Detersive Surfactant

The personal care compositions comprise a detersive surfactant system. The detersive surfactant system is included to provide cleaning performance to the composition. The detersive surfactant system comprises at least one anionic surfactant, and optionally an amphoteric surfactant, a zwitterionic surfactant, a cationic surfactant, a nonionic surfactant, or a combination thereof. Such surfactants should be physically and chemically compatible with the essential components described herein, or should not otherwise unduly impair product stability, aesthetics, or performance.

Suitable anionic surfactant components for use in the personal care composition herein include those that are known for use in hair care or other personal care compositions. The concentration of the anionic surfactant system in the personal care composition should be sufficient to provide the desired cleaning and lather performance, and generally ranges from about 5% to about 50%, preferably from about 8% to about 30%, more preferably from about 10% to about 25%, by weight, of the composition.

In considering the performance characteristics of a personal care composition, such as coacervate formation, wet conditioning performance, dry conditioning performance, and conditioning agent deposition on hair, it is desirable to optimize the levels and types of surfactants in order to maximize the performance potential of polymer systems. In one embodiment, the anionic surfactant system for use in the personal care compositions have an ethoxylate level and an anion level, wherein the ethoxylate level is from about 1 to about 10, and wherein the anion level is from about 1 to about 10. The combination of such an anionic surfactant system with the synthetic copolymer provides enhanced deposition of conditioning agents to hair and/or skin without reducing cleansing or lathering performance.

An optimal ethoxylate level is calculated based on the stoichiometry of the surfactant structure, which in turn is based on a particular molecular weight of the surfactant where the number of moles of ethoxylation is known. Likewise, given a specific molecular weight of a surfactant and an anionization reaction completion measurement, the anion level can be calculated. Analytical techniques have been developed to measure ethoxylation or anionization within surfactant systems. The Level of Ethoxylate and the Level of Anion representative of a particular surfactant system are calculated from the percent ethoxylation and percent anion of individual surfactants in the following manner:
Level of Ethoxylate in a composition=percent ethoxylation multiplied by percent active ethoxylated surfactant (based upon the total weight of the composition).
Level of Anion in a composition=percent anion in ethoxylated surfactant multiplied by percent active ethoxylated surfactant (based upon the total weight of the composition) plus percent anion in non-ethoxylated surfactant multiplied by percent active non-ethoxylated surfactant (based upon the total weight of the composition).

If a composition comprises two or more surfactants having different respective anions (e.g., surfactant A has a sulfate group and surfactant B has a sulfonate group), the Level of Anion in the composition is the sum of the molar levels of each respective anion as calculated above.

Sample Calculation:
Example I shows an ethoxylated surfactant that contains 0.294321% ethoxylate and 0.188307% sulfate as the anion and a non-ethoxylated surfactant that contains 0.266845% sulfate as an anion.
Level of Ethoxylate in Example 1=0.294321 multiplied by 6 (% active ethoxylated surfactant). Thus, the Level of Ethoxylate in the composition of Example 1 is approximately 1.77.
Level of Anion in Example I=0.188307 multiplied by 6 (% active ethoxylated surfactant) plus 0.266845 multiplied by 10 (% active non-ethoxylated surfactant). Thus, the Level of Anion in the composition of Example I is approximately 3.80.

In one embodiment, the detersive surfactant system comprises at least one anionic surfactant comprising an anion selected from the group consisting of sulfates, sulfonates, sulfosuccinates, isethionates, carboxylates, phosphates, and phosphonates. Preferably, the anion is a sulfate.

Preferred anionic surfactants suitable for use in the personal care compositions are alkyl sulfates and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen.

Preferably, R has from about 8 to about 18 carbon atoms, more preferably from about 10 to about 16 carbon atoms, even more preferably from about 12 to about 14 carbon atoms, in both the alkyl sulfates and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with from about 0 to about 10, preferably from about 2 to about 5, more preferably about 3, molar proportions of ethylene oxide, and the resulting mixture of molecular species having, for example, an average of 3 moles of ethylene oxide per mole of alcohol is sulfated and neutralized.

Specific non-limiting examples of alkyl ether sulfates which may be used in the personal care compositions comprise sodium and ammonium salts of coconut alkyl triethylene glycol ether sulfate, tallow alkyl triethylene glycol ether sulfate, and tallow alkyl hexa-oxyethylene sulfate. Highly preferred alkyl ether sulfates are those comprising a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also comprises from about 0% to about 20% by weight C₁₂₋₁₃ compounds; from about 60% to about 100% by weight of C₁₄₋₁₅₋₁₆ compounds; from about 0% to about 20% by weight of C₁₇₋₁₈₋₁₉ compounds; from about 3% to about 30% by weight of compounds having a degree of ethoxylation of 0; from about 45% to about 90% by weight of compounds having a degree of ethoxylation from about 1 to about 4; from about 10% to about 25% by weight of compounds having a degree of ethoxylation from about 4 to about 8; and from about 0.1% to about 15% by weight of compounds having a degree of ethoxylation greater than about 8.

Examples of anionic surfactants for use in the personal care compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and combinations thereof.

In addition to the sulfates, isethionates, sulfonates, sulfosuccinates described above, other potential anions for the anionic surfactant include phosphonates, phosphates, and carboxylates.

The personal care compositions may also comprise one or more additional surfactants selected from the group consisting of amphoteric surfactants, zwitterionic surfactants, cationic surfactants, and nonionic surfactants. Suitable amphoteric, zwitterionic, cationic, or nonionic surfactants for use in the personal care compositions herein include those which are known for use in hair care or other personal care compositions. The concentration of such surfactants preferably ranges from about 0.5% to about 20%, preferably from about 1% to about 10%, by weight of the composition. Non-limiting examples of suitable surfactants are described in U.S. Pat. Nos. 5,104,646 and 5,106,609, both to Bolich, Jr. et al.

Additional Surfactants

Zwitterionic or Amphoteric Surfactant

Suitable amphoteric or zwitterionic surfactants for use in the personal care composition herein include those which are known for use in hair care or other personal care compositions.

Amphoteric surfactants suitable for use in the personal care composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Preferred amphoteric surfactants for use in the personal care compositions comprise cocoamphoacetate, cocoamphodiacetate, lauroamphoacetate, lauroamphodiacetate, lauramine oxide, and mixtures thereof.

Zwitterionic surfactants suitable for use in the personal care composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines (i.e., cocoamidopropyl betaine, cocobetaine), are preferred.

The personal care compositions may further comprise additional surfactants for use in combination with the surfactant component described hereinbefore. Other suitable anionic surfactants are the water-soluble salts of organic, sulfuric acid reaction products conforming to the formula [R¹—SO₃-M] where R¹ is a straight or branched chain, saturated, aliphatic hydrocarbon radical having from about 8 to about 24, preferably from about 10 to about 18 carbon atoms; and M is a cation described hereinbefore. Non limiting examples of such surfactants are the salts of an organic sulfuric acid reaction product of a hydrocarbon of the methane series, including iso-, neo-, and n-paraffins, having from about 8 to about 24 carbon atoms, preferably about 12 to about 18 carbon atoms and a sulfonating agent, e.g., SO₃, H₂SO₄, obtained according to known sulfonation methods, including bleaching and hydrolysis. Preferred are alkali metal and ammonium sulfonated C₁₀ to C₁₈ n-paraffins.

Still other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil.

Other anionic surfactants suitable for use in the personal care compositions are the succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having from about 10 to about 24 carbon atoms, preferably from about 12 to about 16 carbon atoms. Preferably, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha-olefin sulfonate mixture is described in U.S. Pat. No. 3,332,880.

Another class of anionic surfactants suitable for use in the personal care compositions are the beta-alkyloxy alkane sulfonates. These surfactants conform to the Formula II:

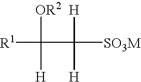

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore. Preferred anionic surfactants for use in the personal care compositions include sodium tridecyl benzene sulfonate, sodium dodecyl benzene sulfonate.
Amides, including alkanol amides, are the condensation products of fatty acids with primary and secondary amines or alkanolamines to yield products of the general Formula III:

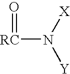

wherein RCO is a fatty acid radical and R is C₈₋₂₀; X is an alkyl, aromatic or alkanol (CHR′CH₂OH wherein R′ is H or C₁₋₆ alkyl); Y is H, alkyl, alkanol or X. Suitable amides include, but are not limited to cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA, PPG-2 Hydroxyethyl cocoamide, and PPG-2-Hydroxyethyl Coco/Isostearamide. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

Suitable optional surfactants include nonionic surfactants. Any such surfactant known in the art for use in hair or personal care products may be used, provided that the optional additional surfactant is also chemically and physically compatible with the essential components of the personal care composition, or does not otherwise unduly impair product performance, aesthetics or stability. The concentration of the optional additional surfactants in the personal care composition may vary with the cleansing or lather performance desired, the optional surfactant selected, the desired product concentration, the presence of other components in the composition, and other factors well known in the art.

Non limiting examples of other surfactants suitable for use in the personal care compositions are described in McCutcheon's, Emulsifiers and Detergents, 1989 Annual, published by M.C. Publishing Co., and U.S. Pat. Nos. 3,929,678; 2,658,072; 2,438,091; 2,528,378.

Aqueous Carrier

The personal care compositions include an aqueous carrier. The level and species of the carrier are selected according to the compatibility with other components and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Additional Cationic Polymers

In order to adjust rinse feel for specific consumer groups, one embodiment comprises blends of the synthetic copolymer with other traditional polymers such as cationic celluloses, cationic guars, cationic starches, or even other cationic synthetic polymers.

Cellulose or Guar Cationic Deposition Polymers

The personal care compositions may also comprise cellulose or guar cationic deposition polymers. Generally, such cellulose or guar cationic deposition polymers may be present at a concentration from about 0.05% to about 5%, by weight of the composition. Suitable cellulose or guar cationic deposition polymers have a molecular weight of greater than about 5,000. Additionally, such cellulose or guar deposition polymers have a charge density from about 0.5 meq/g to about 4.0 meq/g at the pH of intended use of the personal care composition, which pH will generally range from about pH 3 to about pH 9, preferably between about pH 4 and about pH 8. The pH of the compositions is measured neat.

Suitable cellulose or guar cationic polymers include those which conform to the following formula:

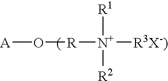

wherein A is an anhydroglucose residual group, such as a cellulose anhydroglucose residual; R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose or guar cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA).

Other suitable cationic deposition polymers include cationic guar gum derivatives, such as guar hydroxypropyltrimonium chloride, specific examples of which include the Jaguar series (preferably Jaguar C-17®) commercially available from Rhone-Poulenc Incorporated, and further including Jaguar C-500, commercially available from Rhodia.

Cationic Deposition Polymers

In addition to the synthetic cationic copolymers of the present invention, the personal care compositions herein may also comprise additional synthetic cationic deposition polymers. Generally, such synthetic cationic deposition polymers may be present at a concentration from about 0.025% to about 5%, by weight of the composition. Such synthetic cationic deposition polymers have a molecular weight from about 1,000 to about 5,000,000. Additionally, such synthetic cationic deposition polymers have a charge density from about 0.5 meq/g to about 10 meq/g.

Suitable synthetic cationic deposition polymers include those which are water-soluble or dispersible, cationic, non-crosslinked, conditioning copolymers comprising: (i) one or more cationic monomer units; and (ii) one or more nonionic monomer units or monomer units bearing a terminal negative charge; wherein said copolymer has a net positive charge, a cationic charge density of from about 0.5 meq/g to about 10 meg/g, and an average molecular weight from about 1,000 to about 5,000,000.

Non-limiting examples of suitable synthetic cationic deposition polymers are described in U.S. Patent Publication No. 2003/0223951 A1 to Geary et al.

Cationically Modified Starch Polymer

In addition to the synthetic cationic copolymers of the present invention, the personal care compositions herein may also comprise additional water-soluble cationically modified starch polymers. As used herein, the term “cationically modified starch” refers to a starch to which a cationic group is added prior to degradation of the starch to a smaller molecular weight, or to a starch to which a cationic group is added after modification of the starch to a desired molecular weight. The definition of the term “cationically modified starch” also includes amphoterically modified starch. The term “amphoterically modified starch” refers to a starch hydrolysate to which a cationic group and an anionic group are added.

In one embodiment, the personal care compositions comprise cationically modified starch polymers at a range of about 0.01% to about 10%, and more preferably from about 0.05% to about 5%, by weight of the composition.

The cationically modified starch polymers for use in the personal care compositions of the present invention have a molecular weight from about 850,000 to about 15,000,000 and more preferably from about 900,000 to about 5,000,000. As used herein, the term “molecular weight” refers to the weight average molecular weight. The weight average molecular weight may be measured by gel permeation chromatography (“GPC”) using an Alliance HPLC (Waters 2695 Separation Module) with two hydrogel columns in series (Waters Ultrahydrogel Linear 6-13 um, 7.8×300 nm GPC column, part number 011545) at a column temperature of 30° C. and at a flow rate of 0.9 ml/min, and using a Viscotek Model 300 TDA (triple detector array), light scattering detector (single angle, 90°), viscosity detector, and refractive index detector, all at detector temperatures of 30° C., with a method created by using pullulan narrow standard P-800 from American Polymer Standards Corporation (M_{w}=788,000), with an injection volume of 25 to 100 μl, and using a dn/dc of 0.147. Additional details on measuring the weight average molecular weight according to a GPC method are described in U.S. Patent Publication No. 2003/0154883 A1, entitled “Non-Thermoplastic Starch Fibers and Starch Composition for Making Same.”

In one embodiment, the personal care compositions of the present invention include cationically modified starch polymers which have a charge density from about 0.2 meq/g to about 5 meq/g, and more preferably from about 0.2 meq/g to about 2 meq/g. The chemical modification to obtain such a charge density includes, but is not limited to, the addition of amino and/or ammonium groups into the starch molecules.

Oily Conditioning Agent

In a preferred embodiment, the personal care compositions comprise one or more oily conditioning agents. Oily conditioning agents include materials which are used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The oily conditioning agents useful in the personal care compositions typically comprise a water-insoluble, water-dispersible, non-volatile, liquid that forms emulsified, liquid particles. Suitable oily conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

One or more oily conditioning agents are typically present at a concentration from about 0.01% to about 10%, preferably from about 0.1% to about 8%, more preferably from about 0.2% to about 4%, by weight of the composition.

In a preferred embodiment, the ratio of oily conditioning agent to synthetic cationic polymer is at least about 2:1

Silicone Conditioning Agent

The oily conditioning agents of the personal care compositions are preferably a water-insoluble silicone conditioning agent. The silicone conditioning agent may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the personal care compositions preferably have a viscosity, as measured at 25° C., from about 20 to about 2,000,000 centistokes (“csk”), more preferably from about 1,000 to about 1,800,000 csk, even more preferably from about 5,000 to about 1,500,000 csk, more preferably from about 10,000 to about 1,000,000 csk.

In one embodiment, the personal care composition is opaque. The personal care composition comprises a non-volatile silicone oil having a particle size as measured in the personal care composition from about 1 μm to about 50 μm. In an embodiment for small particle silicone application to the hair, the personal care composition comprises a non-volatile silicone oil having a particle size as measured in the personal care composition from about 100 nm to about 1 μm. A substantially clear composition embodiment comprises a non-volatile silicone oil having a particle size as measured in the personal care composition of less than about 100 nm.

The transparency of the composition is measured using Ultra-Violet/Visible (UV/VIS) Spectrophotometry, which determines the absorption or transmission of UV/VIS light by a sample. A light wavelength of 600 nm is adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotomer and the percent transmittance is measured by the spectrophotomer at 600 nm.

Non-volatile silicone oils suitable for use in compositions may be selected from organo-modified silicones and fluoro-modified silicones. In one embodiment, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups.

In a preferred embodiment, the non-volatile silicone oil is dimethicone.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone fluids suitable for use in the personal care compositions are disclosed in U.S. Pat. No. 2,826,551; U.S. Pat. No. 3,964,500; U.S. Pat. No. 4,364,837; British Patent No. 849,433, and Silicon Compounds, Petrarch Systems, Inc. (1984).

Organic Conditioning Oils

The oily conditioning agent of the personal care compositions may also comprise at least one organic conditioning oil, either alone or in combination with other conditioning agents, such as the silicones described above.

Hydrocarbon Oils

Suitable organic conditioning oils for use as conditioning agents in the personal care compositions comprise hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include 2,2,4,4,6,6,8,8-dimethyl-10-methylundecane and 2,2,4,4,6,6-dimethyl-8-methylnonane, available from Permethyl Corporation. A preferred hydrocarbon polymer is polybutene, such as the copolymer of isobutylene and butene, which is commercially available as L-14 polybutene from Amoco Chemical Corporation.

Polyolefins

Organic conditioning oils for use in the personal care compositions may also comprise liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

Fatty Esters

Other suitable organic conditioning oils for use as the conditioning agent in the personal care compositions include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the personal care compositions are those known as polyhydric alcohol esters. Such polyhydric alcohol esters include alkylene glycol esters.

Still other fatty esters suitable for use in the personal care compositions are glycerides, including, but not limited to, mono-, di-, and tri-glycerides, preferably di- and tri-glycerides, more preferably triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones. Specific non-limiting examples of suitable fluorinated compounds include the Fomblin product line from Ausimont which includes HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

Fatty Alcohols

Other suitable organic conditioning oils for use in the personal care compositions comprise fatty alcohols having at least about 10 carbon atoms, more preferably about 10 to about 22 carbon atoms, most preferably about 12 to about 16 carbon atoms. Also suitable for use in the personal care compositions are alkoxylated fatty alcohols which conform to the general formula:

CH₃(CH₂)ₙCH₂(OCH₂CH₂)ₚOH

wherein n is a positive integer having a value from about 8 to about 20, preferably about 10 to about 14, and p is a positive integer having a value from about 1 to about 30, preferably from about 2 to about 23.

Alkyl Glucosides and Alkyl Glucoside Derivatives

Suitable organic conditioning oils for use in the personal care compositions comprise alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

Other Conditioning Agents

Quaternary Ammonium Compounds

Suitable quaternary ammonium compounds for use as conditioning agents in the personal care compositions comprise hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in a composition of comprise Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M, and mixtures thereof.

Glycerin may also be used as conditioning agent in the personal care compositions. In one embodiment of the present invention, glycerin may be present in a range from about 0.01% to about 10%. In a further embodiment of the present invention, glycerin may be present in a range from about 0.1% to about 5%. In yet a further embodiment of the present invention, glycerin may be present in a range from about 2% to about 4%.

Additional Components

The personal care compositions may further comprise one or more additional components known for use in hair care or personal care products, provided that the additional components are physically and chemically compatible with the essential components described herein, or do not otherwise unduly impair product stability, aesthetics or performance. Individual concentrations of such additional components may range from about 0.001% to about 10% by weight of the personal care compositions.

Non-limiting examples of additional components for use in the composition include natural cationic deposition polymers, synthetic cationic deposition polymers, anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

Anti-Dandruff Actives

The compositions of the present invention may also contain an anti-dandruff agent. Suitable, non-limiting examples of anti-dandruff particulates include: pyridinethione salts, zinc carbonate, azoles, such as ketoconazole, econazole, and elubiol, selenium sulfide, particulate sulfur, salicylic acid and mixtures thereof. A typical anti-dandruff particulate is pyridinethione salt. Such anti-dandruff particulate should be physically and chemically compatible with the components of the composition, and should not otherwise unduly impair product stability, aesthetics or performance.

a. Pyridinethione Salts

Pyridinethione anti-dandruff particulates, especially 1-hydroxy-2-pyridinethione salts, are suitable particulate anti-dandruff agents for use in compositions of the present invention. The concentration of pyridinethione anti-dandruff particulate typically ranges from about 0.01% to about 5%, by weight of the composition, generally from about 0.1% to about 3%, commonly from about 0.1% to about 2%. Suitable pyridinethione salts include those formed from heavy metals such as zinc, tin, cadmium, magnesium, aluminum and zirconium, generally zinc, typically the zinc salt of 1-hydroxy-2-pyridinethione (known as “zinc pyridinethione” or “ZPT”), commonly 1-hydroxy-2-pyridinethione salts in platelet particle form, wherein the particles have an average size of up to about 20μ, typically up to about 5μ, commonly up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

b. Anti-Microbial Actives

In addition to the anti-dandruff active selected from polyvalent metal salts of pyrithione, the present invention may further comprise one or more anti-fungal or anti-microbial actives in addition to the metal pyrithione salt actives. Suitable anti-microbial actives include coal tar, sulfur, charcoal, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseo fulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Typical anti-microbials include itraconazole, ketoconazole, selenium sulphide and coal tar.

i. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, typically from about 0.1% to about 3%, and commonly from about 0.3% to about 2%, by weight of the composition. Especially common for use herein is ketoconazole.

ii. Selenium Sulfide

Selenium sulfide is a particulate anti-dandruff agent suitable for use in the anti-microbial compositions of the present invention, effective concentrations of which range from about 0.1% to about 4%, by weight of the composition, typically from about 0.3% to about 2.5%, commonly from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), typically less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

iii. Sulfur

Sulfur may also be used as a particulate anti-microbial/anti-dandruff agent in the anti-microbial compositions of the present invention. Effective concentrations of the particulate sulfur are typically from about 1% to about 4%, by weight of the composition, typically from about 2% to about 4%.

iv. Keratolytic Agents

The present invention may further comprise one or more keratolytic agents such as Salicylic Acid.

The present invention may also comprise combination of anti-microbial actives. Such combinations may include octopirox and zinc pyrithione, pine tar and sulfur combinations, salicylic acid and zinc pyrithione combinations, salicylic acid and elubiol combinations, zinc pyrithione and elubiol combinations, octopirox and climbasole combinations, and salicylic acid and octopirox combinations and mixtures thereof.

Zinc-Containing Layered Material

In an embodiment of the present invention, the composition may include an effective amount of a zinc-containing layered material. Preferred embodiments of the present invention include from about 0.001% to about 10% of a zinc-containing layered material; more preferably from about 0.01% to about 7%; more preferably still from about 0.1% to about 5%.

Examples of zinc-containing layered materials useful in certain embodiments of the present invention include the following:

Zinc-containing layered structures are those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or be components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}.nH2O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y)/n}.nH₂O where the two metal ion may be different; if they are the same and represented by zinc, the formula simplifies to [Zn₁₊ₓ(OH)₂]^{2x+}2xA⁻.nH₂O. This latter formula represents (where x=0.4) common materials such as zinc hydroxychloride and zinc hydroxynitrate. These are related to hydrozincite as well wherein a divalent anion replace the monovalent anion. These materials can also be formed in situ in a composition or in or during a production process.

These classes of ZLM's represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice

It has been found, in accordance with an embodiment of the present invention, that anti-dandruff efficacy can be dramatically increased in topical compositions by the combination of an effective amount of a zinc-containing layered material wherein the zinc-containing layered material has a specified zinc lability within a surfactant system. Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum.

Labile zinc is maintained by choice of an effective zinc-containing layered material or formation of an effective zinc-containing layered material in-situ by known methods.

It has been found, in accordance with an embodiment of the present invention, that anti-dandruff efficacy can be dramatically increased in topical compositions by the use of polyvalent metal salts of pyrithione, such as zinc pyrithione, in combination with zinc-containing layered materials. Therefore an embodiment of the present invention provides topical compositions with improved benefits to the skin and scalp (e.g., improved antidandruff efficacy).

An embodiment of the present invention provides a stable composition for zinc-containing layered material dispersion where the zinc source resides in a particulate form. It has been shown to be challenging to formulate aqueous systems containing a zinc-containing layered material, due to the zinc-containing layered material's unique physical and chemical properties. Zinc-containing layered material may have a high density (approximately 3 g/cm³), and needs to be evenly dispersed throughout the product and so it will not aggregate or settle. Zinc-containing layered material also has a very-reactive surface chemistry as well as the propensity to dissolve in systems with pH values below 6.5.

A zinc-containing layered material with a solubility of less than 25% will have a measurable % soluble zinc value below a threshold value determined by the weight percent and molecular weight of the zinc compound. The theoretical threshold value can be calculated by the following equation:

$$\frac{\begin{matrix}0.25 * wt . \% Zn Compound in Composition * \\ moles of Zinc in Compound * 65.39 ( MW of Zn )\end{matrix}}{MW of Zn Compound}$$

In an embodiment of the present invention, wherein the composition comprises a ZLM, the pH may be greater than about 6.5; further, the pH may be in a range from about 6.5 to about 12, preferably from about 6.8 to about 9.5, more preferably from about 6.8 to about 8.5.

Particle Size of ZLM

In an embodiment of the present invention, it is has been found that a smaller particle size is inversely proportional to relative zinc lability

D(90) is the particle size which corresponds to 90% of the amount of particles are below this size. In an embodiment of the present invention, the zinc-containing layered material may have a particle size distribution wherein 90% of the particles are less than about 50 microns. In a further embodiment of the present invention, the zinc-containing layered material may have a particle size distribution wherein 90% of the particles are less than about 30 microns. In yet a further embodiment of the present invention, the zinc-containing layered material may have a particle size distribution wherein 90% of the particles are less than about 20 microns.

Surface Area of ZLM

In an embodiment of the present invention, there may be a direct relationship between surface area and relative zinc lability.

Increased particle surface area generally increases zinc lability due to kinetic factors. Particulate surface area can be increased by decreasing particle size and/or altering the particle morphology to result in a porous particle or one whose overall shape deviates geometrically from sphericity.

In an embodiment of the present invention, the basic zinc carbonate may have a surface area of greater than about 10 m²/gm. In a further embodiment, the basic zinc carbonate may have a surface area of greater than about 20 m²/gm. In yet a further embodiment of the present invention, the basic zinc carbonate may have a surface area of greater than about 30 m²/gm.

Coordinating Compound Having a Log Zn Binding Constant

In a further embodiment of the present invention, the composition further comprises a coordinating compound with a Log Zn binding constant in a range sufficient to maintain zinc bioavailability. Preferably, such a coordinating compound has a Log Zn binding constant less than about 6, preferably less than about 5, more preferable less than about 4, and greater than about 0.5. Preferably such a coordinating compound is an organic acid, strong mineral acid, or coordinating species. Preferred examples of such coordinating compounds include the following (respective Log Zn Binding Constant indicated in parenthesis): EDTA (16.5), EDDS (13.5), EDDA (11.1), NTA (10.7), Xylenol Orange (10.3), Cysteine (9.1), Cystine (6.7), Aspartic Acid (Aspartate) (5.9), Glycine (5.0), Citric Acid (Citrate) (4.8), Glutamic Acid (4.5), Methionine (4.4), Arginine (4.2), Carbonic Acid (Carbonate) (3.9), Ornithine (3.8), Tatronic Acid (Tartrate) (3.2), Malic Acid (Malate) (2.9), Malonic Acid (Malonate) (2.9), Tartaric Acid (Tartrate) (2.7), Adipic Acid (Adipate) (2.6), Phosphoric Acid (Phosphate) (2.4), Phthalic Acid (Phthalate) (2.2), Glycolic Acid (Glycolate) (2.0), Lactic Acid (Lactate) (1.9), Succinic Acid (Succinate) (1.8), Acetic Acid (Acetate) (1.0), Sulfuric Acid (Sulfate) (0.9), Boric Acid (Borate) (0.9), Formic Acid (Formate) (0.6), Chloride (−0.3).

Pyrithione or a Polyvalent Metal Salt of Pyrithione

In an embodiment, the present invention may comprise pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

It is further contemplated that when ZPT is used as the anti-microbial particulate in the anti-microbial compositions herein, that an additional benefit of hair growth or re-growth may be stimulated or regulated, or both, or that hair loss may be reduced or inhibited, or that hair may appear thicker or fuller.

Zinc pyrithione may be made by reacting 1-hydroxy-2-pyridinethione (i.e., pyrithione acid) or a soluble salt thereof with a zinc salt (e.g. zinc sulfate) to form a zinc pyrithione precipitate, as illustrated in U.S. Pat. No. 2,809,971.

Preferred embodiments include from about 0.01% to about 5% of a pyrithione or polyvalent metal salt of a pyrithione; more preferably from about 0.1% to about 2%.

In embodiments having a zinc-containing layered material and a pyrithione or polyvalent metal salt of pyrithione, the ratio of zinc-containing layered material to pyrithione or a polyvalent metal salt of pyrithione is preferably from 5:100 to 10:1; more preferably from about 2:10 to 5:1; more preferably still from 1:2 to 3:1.

Particles

The personal care compositions may also comprise particles. Useful particles can be inorganic, synthetic, or semi-synthetic. In the present invention, it is preferable to incorporate no more than about 20%, more preferably no more than about 10% and even more preferably no more than 2%, by weight of the composition, of particles. In one embodiment, the particles have an average mean particle size of less than about 300 μm.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles comprise silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

Opacifying Agents

The personal care compositions may also comprise one or more opacifying agents. Opacifying agents are typically used in cleansing compositions to impart desired aesthetic benefits to the composition, such as color or pearlescence. In the present invention, it is preferable to incorporate from about 0.01% to about 20%, more preferably from about 0.01% to about 10% and even more preferably from about 0.01% to about 2%, by weight of the composition, of opacifying agents.

Suitable opacifying agents include, for example, fumed silica, polymethylmethacrylate, micronized Teflon®, boron nitride, barium sulfate, acrylate polymers, aluminum silicate, aluminum starch octenylsuccinate, calcium silicate, cellulose, chalk, corn starch, diatomaceous earth, Fuller's earth, glyceryl starch, hydrated silica, magnesium carbonate, magnesium hydroxide, magnesium oxide, magnesium trisilicate, maltodextrin, microcrystalline cellulose, rice starch, silica, titanium dioxide, zinc laurate, zinc myristate, zinc neodecanoate, zinc rosinate, zinc stearate, polyethylene, alumina, attapulgite, calcium carbonate, calcium silicate, dextran, nylon, silica silylate, silk powder, soy flour, tin oxide, titanium hydroxide, trimagnesium phosphate, walnut shell powder, or mixtures thereof. The above mentioned powders may be surface treated with lecithin, amino acids, mineral oil, silicone oil, or various other agents either alone or in combination, which coat the powder surface and render the particles hydrophobic in nature.

The opacifying agents may also comprise various organic and inorganic pigments. The organic pigments are generally various aromatic types including azo, indigoid, triphenylmethane, anthraquinone, and xanthine dyes. Inorganic pigments include iron oxides, ultramarine and chromium or chromium hydroxide colors, and mixtures thereof.

Suspending Agents

The personal care compositions may further comprise a suspending agent at concentrations effective for suspending water-insoluble material in dispersed form in the compositions or for modifying the viscosity of the composition. Such concentrations generally range from about 0.1% to about 10%, preferably from about 0.3% to about 5.0%, by weight of the composition, of suspending agent.

Suspending agents useful herein include anionic polymers and nonionic polymers. Useful herein are vinyl polymers such as cross linked acrylic acid polymers with the CTFA name Carbomer.

Paraffinic Hydrocarbons

The personal care compositions may also comprise one or more paraffinic hydrocarbons. Paraffinic hydrocarbons suitable for use in compositions of the present invention include those materials which are known for use in hair care or other personal care compositions, such as those having a vapor pressure at 1 atm of equal to or greater than about 21° C. (about 70° F.). Non-limiting examples include pentane and isopentane.

Propellants

The personal care compositions may comprise one or more propellants. Propellants suitable for use in compositions of the present invention include those materials which are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

Mono or Divalent Salt

The personal care compositions may further comprise a mono or divalent salt, which acts as a source of entropy to assist in coacervate formation. Salt allows more contacts to be made between the polymer and surfactant, which increases the formation of coacervate. By the term “coacervate initiator,” as used herein, means salt capable of inducing the formation of coacervates when combined with compositions comprising an anionic detersive surfactant component surfactant system and the synthetic cationic polymer.

Surfactant salts themselves are not included in the present salt definition but other salts are. Suitable salts comprise chlorides, phosphates, sulfates, nitrates, citrates and halides. The counter ions of such salts can be, but are not limited to, sodium, potassium, ammonium, magnesium, zinc or other mono and divalent cation. Salts most preferred for use in the compositions of the present invention include sodium chloride, ammonium chloride, sodium citrate, magnesium chloride, and magnesium sulfate. It is recognized that these salts may serve as thickening aids or buffering aids in addition to their role as a coacervate initiator. The amount of coacervate initiator comprising the salt and/or the optional surfactant will vary with the type of surfactant and polymer, but is preferably present at a level of from about 0.01% to about 5%, more preferably from about 0.05% to about 3.5%, and still more preferably from about 0.1% to about 2%.

Other Optional Components

The personal care compositions may contain fragrance.

The personal care compositions may also comprise water-soluble and water-insoluble vitamins such as vitamins B1, B2, B6, B12, C, pantothenic acid, pantothenyl ethyl ether, panthenol, biotin and their derivatives, and vitamins A, D, E, and their derivatives. The compositions of the present invention may also contain water-soluble and water-insoluble amino acids such as asparagine, alanine, indole, glutamic acid and their salts, and tyrosine, tryptamine, lysine, histadine and their salts.

The compositions of the present invention may also contain chelating agents.

The compositions of present invention may further comprise materials useful for hair loss prevention and hair growth stimulants or agents.

Method for Measuring Lather Volume

The potential for the personal care compositions disclosed herein to generate lather is measured via the SITA Foam Tester (model: R-2000) made by SITA Messtechnik GmbH (Germany). The SITA Foam Tester R-2000 utilizes a patented rotor of defined geometry for foam generation. The rotor mechanically inserts air bubbles into the liquid. The foam volume is measured by an array of sensor needles, which scans the foam surface. Using an array of sensor needles permits exact measurement of the foam volume even with uneven foam surfaces. The output is given as average millimeters of foam height per measure. Foam height measurements are taken every 10 seconds. The stir count and stir time refer to the input in the SITA program. The SITA program stirs for 10 sec then a measure is taken, then stirs again for 10 seconds a measure is taken—this occurs 12 times in total (stirred for 12 separate 10 sec. intervals). The Stir Count, as used herein, means the total number of stirring intervals in one test. It has been found that the 40 second measurement, the fourth total measurement, is particularly relevant to the consumer usage experience. At the 40 second measurement, foam heights of at least about 50 millimeters are particularly desirable, even more preferred are foam heights of at least about 100 millimeters. To measure these values, standard manufacturer's methods are followed for operation of the equipment with the following requirements:

Instrument Settings/Measurement Parameters:

| | |
|---|---|
| Volume of Water (ml) | 300 |
| Mixing Rotor Speed (rpm) | 1000 |
| Stir Count | 12 |
| Stir Time (sec) | 10 |

Water/Product/Soil load:

300 gm of 7 grain hardness water at 100° F.
0.5 ml test product (cleansing composition)
0.05 ml Extra Virgin, first cold pressed Olive Oil (simulates sebum)

The above materials may either be premixed prior to being fed into the SITA unit or the water may be metered in and the test product and soil may be injected into the SITA vessel either at the top of the vessel or through the injection port.

Method for Assessment of Zinc Lability in Zinc-Containing Products

Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum.

Zinc lability is assessed by combining a diluted zinc-containing solution or dispersion with the metallochromic dye xylenol orange (XO) and measurement of the degree of color change under specified conditions. The magnitude of color formation is proportional to the level of labile zinc. The procedure developed has been optimized for aqueous surfactant formulations but may be adapted to other physical product forms as well.

A spectrophotometer is used to quantify the color change at 572 nm, the wavelength of optimum color change for XO. The spectrophotometer is set to zero absorbance at 572 nm utilizing a product control as close in composition to the test product except excluding the potentially labile form of zinc. The control and test products are then treated identically as follows. A 5011 product sample is dispensed into ajar and 95 ml of deaerated, distilled water are added and stirred. 5 mL of a 23 mg/mL xylenol orange stock solution at pH 5.0 is pipetted into the sample jar; this is considered time 0. The pH is then adjusted to 5. 50±0.01 using dilute HCl or NaOH. After 10.0 minutes, a portion of the sample is filtered (0.45μ) and the absorbance measured at 572 nm. The measured absorbance is then compared to a separately measured control to determine the relative zinc lability (zero TO 100%). The 100% lability control is prepared in a matrix similar to the test products but utilizing a soluble zinc material (such as zinc sulfate) incorporated at an equivalent level on a zinc basis. The absorbance of the 100% lability control is measured as above for the test materials. The relative zinc lability is preferably greater than about 15%, more preferably greater than about 20%, and even more preferably greater than about 25%.

Using this methodology, the below examples demonstrate a material (basic zinc carbonate) that has intrinsically high lability in an anionic surfactant system compared to one (ZnO) with low intrinsic lability.

| | | Relative Zinc | |
|---|---|---|---|
| | Relative Zinc | Lability (%) | |
| | Lability (%) | In Simple Surfactant | |
| | In Water | System¹ | Lability Benefit |
| Zinc Oxide | 86.3 | 1.5 | NO |
| Basic zinc | 100 | 37 | YES |
| carbonate | | | |

¹Simple surfactant system: 6% sodium lauryl sulfate

Particle Size Determination Method

Particle size analyses on zinc oxide and hydrozincite raw materials are done using the Horiba LA-910 Particle Size Analyzer. The Horiba LA-910 instrument uses the principles of low-angle Fraunhofer Diffraction and Light Scattering to measure the particle size and distribution in a dilute solution of particles. Samples of these two types of raw materials are predispersed in a dilute solution of Lauryl Polyether Alcohol and mixed before introduction to the instrument. On introduction the sample is further diluted and allowed to circulate in the instrument before a measurement is taken. After measurement a calculation algorithm is used to process the data that results in both a particle size and distribution. D(50) is the median particle size or the particle size which corresponds to 50% of the amount of particles are below this size.

D(90) is the particle size which corresponds to 90% of the amount of particles are below this size.
D(10) is the particle size which corresponds to 10% of the amount of particles are below this size.

Using this methodology, the below examples demonstrate the relationship between particle size and relative zinc lability for basic zinc carbonate.

| | | | Relative |
|---|---|---|---|
| | | | Zinc |
| | As | Particle | Lability |
| Source | received/milled¹ | Size (μ)² | (%) |
| Elementis | As received | 4.5 | 51.6 |
| Elementis | Milled | 1.0 | 67.1 |
| Brüggemann | As received | 4.5 | 56.9 |
| Brüggemann | Milled | 1.0 | 76.4 |

¹Milling method
²Particle size Determination

Surface Area Methodology

Surface area analysis is done using the Micromeritics Auto Pore IV. The Micromeritics Auto Pore IV uses the principles of capillary law governing penetration of a non-wetting liquid, more specifically mercury, into small pores to measure the total pore surface area. This law is expressed by the Washburn equation:

D=(1/P)4γ cos φ

where D is pore diameter, P is the applied pressure, γ the surface tension of mercury, and φ the contact angle between the mercury and the sample. The Washburn equation assumes that all pores are cylindrical. Representative surface area measurements were conducted on basic zinc carbonate and are described below.

Results

| Sample | Surface Area (m²/g) |
|---|---|
| Brüggemann Zinc Carbonate¹ | 50.57 |
| Elementis Zinc Carbonate² | 38.0 |

¹Commercially available as Zinc Carbonate AC
²Commerically available as Zinc Carbonate

Method of Making

The compositions of the present invention, in general, may be made by mixing the ingredients together at either room temperature or at elevated temperature, e.g. about 72° C. Heat only needs to be used if solid ingredients are in the composition. The ingredients are mixed at the batch processing temperature. Additional ingredients, including electrolytes, polymers, fragrance, and particles, may be added to the product at room temperature.

Method of Making

The personal care compositions may be made by mixing the ingredients together at either room temperature or at elevated temperature, e.g., about 72° C. Heat only needs to be used if solid ingredients are to be incorporated into the composition. The ingredients are mixed at the batch processing temperature. Additional ingredients, including electrolytes, polymers, fragrance, and particles, may be added to the product at room temperature.

Method of Treating Hair or Skin

The personal care compositions are used in a conventional manner for cleansing and conditioning hair or skin. Generally, a method of treating hair or skin of the present invention comprises applying the personal care composition of the present invention to the hair or skin. More specifically, an effective amount of the personal care composition is applied to the hair or skin, which has preferably been wetted with water, and then the personal care composition is rinsed off. Such effective amounts generally range from about 1 g to about 50 g, preferably from about 1 g to about 20 g. Application to the hair typically includes working the composition through the hair such that most or all of the hair is contacted with the composition.

This method for treating the hair or skin comprises the steps of: (a) applying an effective amount of the personal care composition to the hair or skin, and (b) rinsing the applied areas of hair or skin with water. These steps can be repeated as many times as desired to achieve the desired cleansing and conditioning benefit.

For use in methods of the present invention, the personal care composition may be in various forms, for example, shampoos, body washes, gels, lotions, creams, mousses, and sprays. For some of these forms, the personal care composition may be packaged in a pump-dispenser bottle or in an aerosol container. In other useful forms, the personal care composition may be dried to a film or a powder, or it may be applied to a substrate which is then used for application to the hair or skin.

### Non-Limiting Examples

The compositions illustrated in the following Examples illustrate specific embodiments of the compositions of the present invention, but are not intended to be limiting thereof. Other modifications can be undertaken by the skilled artisan without departing from the spirit and scope of this invention. These exemplified embodiments of the composition of the present invention provide enhanced deposition of conditioning agents to the hair and/or skin.

The compositions illustrated in the following Examples are prepared by conventional formulation and mixing methods, an example of which is described above. All exemplified amounts are listed as weight percents and exclude minor materials such as diluents, preservatives, color solutions, imagery ingredients, botanicals, and so forth, unless otherwise specified.

The following are representative of personal care compositions of the present invention:

| | EXAMPLE COMPOSITION | | |
|---|---|---|---|
| Ingredient | I | II | III |
| Water | q.s. | q.s. | q.s. |
| Guar Hydroxy Propyl Trimonium Chloride (1) | 0.23 | | |
| AM:TRIQUAT Copolymer (2) | 0.08 | 0.2 | 0.2 |
| Sodium Laureth Sulfate (SLE3S - 28% active) (3) | 21.43 | 21.43 | 21.43 |
| Sodium Lauryl Sulfate (SLS - 29% active) (4) | 24.14 | 24.14 | 24.14 |
| Cocoamdopropyl Betaine (5) | 3.33 | 3.33 | 3.33 |
| Ethylene Glycol Disterate(6) | 2 | 2 | 2 |
| 330M silicone (7) | 1.7 | 1.1 | |
| Aminosilicone (8) | | | 1.4 |
| Sodium Chloride (9) | Up to 1.5% | Up to 1.5% | Up to 1.5% |
| Fragrance | 0.75 | 0.75 | 0.75 |
| Preservatives, pH adjusters | Up to 1.3% | Up to 1.3% | Up to 1.3% |
| ZPT (10) | 1 | 1 | 1 |
| Zinc Hydroxy Carbonate (11) | 1.61 | 1.61 | 1.61 |
| Sodium Xylenesulfonate (12) | Up to 1% | Up to 1% | Up to 1% |
| Calculated: | | | |
| Ethoxylate level | 1.77 | 1.77 | 1.77 |
| Sulfate level | 3.00 | 3.00 | 3.00 |

(1) Jaguar C500 supplier: Rhodia.
(2) Copolymer of Acrylamide(AM) and TRIQUAT, MW = 1,000,000; CD = 1.6 meq./gram;
AM:TRIQUAT ratio = 95:5
(3) Sodium Laureth (3M ethylene oxide) Sulfate at 28% active, supplier: P&G
(4) Sodium Lauryl Sulfate at 29% active, supplier: P&G
(5) Tegobetaine F-B, 30% active, supplier: Goldschmidt Chemical
(6) Ethylene Glycol Disterate at 100% active, supplier: Goldschmidt Chemical
(7) 330 M silicone, 100% active, supplier: Momentive (silicone used by P&G to make a
70% active, 30 um emulsion)
(8) Aminosilicone; supplier General Electric; terminal aminopropyl substitution, viscosity 350,000,
D ~1600, M′ = 2, particle size 3 mm
(9) Sodium Chloride USP (food grade)
(10) ZPT having an average particle size of about 2.5 mm, available from Arch/Olin.
(11) Zinc Hydroxy Carbonate available from Bruggeman
(12) Sodium Xylene Sulfonate, supplier: Stepan

| | EXAMPLE COMPOSITION | | | | | | |
|---|---|---|---|---|---|---|---|
| Ingredient | IV | V | VI | VII | VIII | IX | X |
| Water | q.s. | q.s. | q.s. | q.s. | q.s. | q.s. | q.s. |
| AM:TRIQUAT Copolymer¹ | 0.25 | — | 0.25 | — | 0.25 | — | 0.25 |
| AM:TRIQUAT Copolymer² | — | 0.25 | — | 0.25 | — | 0.25 | — |
| Sodium Laureth Sulfate (SLE3S)³ | — | 6.00 | 8.00 | 8.00 | 10.00 | 12.00 | 14.00 |
| Sodium Lauryl Sulfate (SLS)⁴ | — | 10.00 | — | 6.00 | — | — | — |
| Ammonium Laureth Sulfate | 6.00 | — | — | — | — | — | — |
| (ALE3S)⁵ | | | | | | | |
| Ammonium Lauryl Sulfate (ALS)⁶ | 10.00 | — | — | — | — | — | — |
| Cocoamidopropyl Betaine⁷ | — | — | 4.00 | 2.00 | 2.00 | 4.00 | 4.00 |
| PPG-2 Hydroxyethyl | — | 2.00 | 2.00 | 2.00 | 2.00 | 2.00 | 2.00 |
| Coco/Isostearamide⁸ | | | | | | | |
| Sodium Chloride⁹ | 2.50 | 2.25 | 1.50 | 0.50 | 1.00 | — | 1.50 |
| Sodium Xylene Sulfonate¹⁰ | — | — | — | — | — | 0.25 | — |
| Fragrance | 0.70 | 0.70 | 0.70 | 0.70 | 0.70 | 0.70 | 0.70 |
| Preservatives, pH adjusters | Up | Up | Up | Up | Up | Up | Up |
| | to | to | to | to | to | to | to |
| | 1% | 1% | 1% | 1% | 1% | 1% | 1% |
| Calculated: | | | | | | | |
| Ethoxylate Level | 1.77 | 1.77 | 2.35 | 2.35 | 2.94 | 3.53 | 4.12 |
| Sulfate Level | 3.80 | 3.80 | 1.51 | 3.11 | 1.88 | 2.26 | 2.64 |
| Ethoxylate:Sulfate (Ratio) | 0.46 | 0.46 | 1.56 | 0.76 | 1.56 | 1.56 | 1.56 |

¹Copolymer of Acrylamide(AM) and TRIQUAT, MW = 1,000,000; CD = 1.6 meq./
gram; AM:TRIQUAT ratio = 95:5
²Copolymer of Acrylamide(AM) and TRIQUAT, MW = 400,000; CD = 1.6 meq./
gram; AM:TRIQUAT ratio = 95:5
³Sodium Laureth Sulfate, supplier: P&G
⁴Sodium Lauryl Sulfate, supplier: P&G
Ammonium Laureth Sulfate, supplier: P&G
Ammonium Lauryl Sulfate, supplier: P&G
⁵Tegobetaine F-B, supplier: Goldschmidt Chemicals
⁶Promidium 2, supplier Unichema
⁷Sodium Chloride USP (food grade), supplier Morton
⁸Sodium Xylene Sulfonate, supplier: Stepan

| | EXAMPLE COMPOSITION | | | | | | |
|---|---|---|---|---|---|---|---|
| Ingredient | XI | XII | XIII | XIV | XV | XVI | XVII |
| Water | q.s. | q.s. | q.s. | q.s. | q.s. | q.s. | q.s. |
| AM:TRIQUAT Copolymer¹ | 0.25 | — | 0.25 | — | 0.25 | — | 0.25 |
| AM:TRIQUAT Copolymer² | — | 0.25 | — | 0.25 | — | 0.25 | — |
| Sodium Laureth Sulfate (SLE3S)³ | 6.00 | 6.00 | 8.00 | 8.00 | 10.00 | 12.00 | 14.00 |
| Sodium Lauryl Sulfate (SLS)⁴ | 10.00 | 10.00 | — | 6.00 | — | — | — |
| Dimethiconol Microemulsion A⁵ | — | — | — | 1.00 | — | — | — |
| Dimethiconol Microemulsion B⁶ | 1.00 | 1.00 | 1.00 | — | — | — | — |
| Disodium Coco Amphodiacetate⁷ | — | — | 2.00 | 2.00 | 2.00 | 4.00 | 4.00 |
| Cocoamidopropyl Betaine⁸ | — | 2.00 | — | 2.00 | 2.00 | 2.00 | 2.00 |
| PPG-2 Hydroxyethyl | — | 2.00 | — | 2.00 | 2.00 | 2.00 | 2.00 |
| Coco/Isostearamide⁹ | | | | | | | |
| Cocoamide MEA¹⁰ | 1.50 | — | 0.80 | — | — | — | — |
| Sodium Chloride¹¹ | 0.25 | 2.00 | 3.00 | 1.50 | 1.50 | 1.00 | 1.50 |
| Fragrance | 0.70 | 0.70 | 0.70 | 0.70 | 0.70 | 0.70 | 0.70 |
| Preservatives, pH adjusters | Up | Up | Up | Up | Up | Up | Up |
| | to | to | to | to | to | to | to |
| | 1% | 1% | 1% | 1% | 1% | 1% | 1% |
| Calculated: | | | | | | | |
| Ethoxylate Level | 1.77 | 1.77 | 2.35 | 2.35 | 2.94 | 3.53 | 4.12 |
| Sulfate Level | 3.80 | 3.80 | 1.51 | 3.11 | 1.88 | 2.26 | 2.64 |
| Ethoxylate:Sulfate (Ratio) | 0.46 | 0.46 | 1.56 | 0.76 | 1.56 | 1.56 | 1.56 |

¹Copolymer of Acrylamide(AM) and TRIQUAT, MW = 1,000,000; CD = 1.6 meq./
gram; AM:TRIQUAT ratio = 95:5
²Copolymer of Acrylamide(AM) and TRIQUAT, MW = 400,000; CD = 1.6 meq./
gram; AM:TRIQUAT ratio = 95:5
³Sodium Laureth Sulfate, supplier: P&G
⁴Sodium Lauryl Sulfate, supplier: P&G
⁵Dow Corning Silicone Micro-emulsion DC2-1870; Internal Phase Viscosity =
72,000; 30 nm particle size dimethiconol using TEA dodecyl benzene
sulfonate and laureth 23 as primary surfactants
⁶Dow Corning DC 2-1865; Internal Phase Viscosity = 25,000 cps; 25 nm
particle size dimethiconol using TEA dodecyl benzene sulfonate and laureth
23 as primary surfactants
⁷Miranol C2M Conc NP, supplier: Rhodia.
⁸Tegobetaine F-B, supplier: Goldschmidt Chemicals
⁹Promidium 2, supplier Unichema
¹⁰Monamid CMA, supplier Goldschmidt Chemical
¹¹Sodium Chloride USP (food grade), supplier Morton.

| | EXAMPLE COMPOSITION | | | | |
|---|---|---|---|---|---|
| Ingredient | XVIII | XIX | XX | XXI | XXII |
| Water | q.s. | q.s. | q.s. | q.s. | q.s. |
| AM:TRIQUAT Copolymer¹ | 0.25 | 0.25 | 0.25 | — | 0.25 |
| AM:TRIQUAT Copolymer² | — | — | — | 0.25 | — |
| Sodium Laureth Sulfate (SLE3S)³ | — | 10.00 | — | 8.00 | 14.00 |
| Sodium Lauryl Sulfate (SLS)⁴ | — | — | — | 3.00 | — |
| Ammonium Laureth Sulfate (ALE3S)⁵ | 6.00 | — | 14.00 | — | — |
| Ammonium Lauryl Sulfate (ALS)⁶ | 10.00 | — | 2.00 | — | — |
| Dimethicone Emulsion⁵ | 1.00 | 2.00 | 1.00 | 0.50 | 1.00 |
| Disodium Coco Amphodiacetate⁶ | — | 2.00 | — | — | — |
| Cocoamidopropyl Betaine⁷ | — | — | 2.00 | 4.00 | 2.00 |
| PPG-2 Hydroxyethyl Coco/Isostearamide⁸ | 2.00 | 2.00 | 2.00 | — | 2.00 |
| Ethylene Glycol Distearate⁹ | 1.50 | — | — | — | 1.50 |
| Sodium Chloride¹⁰ | 1.25 | 1.25 | 1.25 | 1.00 | 1.50 |
| Fragrance | 0.70 | 0.70 | 0.70 | 0.70 | 0.70 |
| Preservatives, pH adjusters | Up | Up | Up | Up | Up |
| | to | to | to | to | to |
| | 1% | 1% | 1% | 1% | 1% |
| Calculated: | | | | | |
| Ethoxylate Level | 1.77 | 2.94 | 4.12 | 2.35 | 4.12 |
| Sulfate Level | 3.80 | 1.88 | 3.17 | 2.31 | 2.64 |
| Ethoxylate:Sulfate (Ratio) | 0.46 | 1.56 | 1.30 | 1.02 | 1.56 |

¹Copolymer of Acrylamide(AM) and TRIQUAT, MW = 1,000,000; CD = 1.6 meq./
gram; AM:TRIQUAT ratio = 95:5
²Copolymer of Acrylamide(AM) and TRIQUAT, MW = 400,000; CD = 1.6 meq./
gram; AM:TRIQUAT ratio = 95:5
³Sodium Laureth Sulfate, supplier: P&G
⁴Sodium Lauryl Sulfate, supplier: P&G
⁵Dow Corning Dimethicone emulsion DC-1664; 3 micron particle size
⁶Miranol C2M Conc NP, supplier: Rhodia.
⁷Tegobetaine F-B, supplier: Goldschmidt Chemicals
⁸Promidium 2, supplier Unichema
⁹Ethylene Glycol Distearate, EGDS Pure, supplier Goldschmidt Chemical
¹⁰Sodium Chloride USP (food grade), supplier Morton.

| | EXAMPLE COMPOSITION | | | | |
|---|---|---|---|---|---|
| Ingredient | XXIII | XXIV | XXV | XXVI | XXVII |
| Water | q.s. | q.s. | q.s. | q.s. | q.s. |
| AM:TRIQUAT Copolymer¹ | 0.25 | 0.25 | 0.25 | — | 0.25 |
| AM:TRIQUAT Copolymer² | — | — | — | 0.25 | — |
| Sodium Laureth Sulfate (SLE3S)³ | — | 6.00 | 8.00 | 8.00 | — |
| Sodium Lauryl Sulfate (SLS)⁴ | — | 10.00 | 8.00 | 10.00 | — |
| Ammonium Laureth Sulfate (ALE3S)⁵ | 6.00 | — | — | — | 15.00 |
| Ammonium Lauryl Sulfate (ALS)⁶ | 10.00 | — | — | — | 2.00 |
| Dimethicone⁵ | 1.20 | 0.5 | 1.00 | 0.50 | 1.00 |
| Cocoamidopropyl Betaine⁶ | — | — | 2.00 | 1.00 | 5.00 |
| Cocoamide MEA⁷ | 1.5 | 0.75 | — | — | — |
| Ethylene Glycol Distearate⁸ | 1.50 | 1.50 | 1.50 | 1.50 | 1.50 |
| Sodium Chloride⁹ | 1.50 | 1.25 | 1.00 | 1.25 | 1.50 |
| Fragrance | 0.70 | 0.70 | 0.70 | 0.70 | 0.70 |
| Preservatives, pH adjusters | Up to | Up to | Up to | Up | Up |
| | 1% | 1% | 1% | to | to |
| | | | | 1% | 1% |
| Calculated: | | | | | |
| Ethoxylate Level | 1.77 | 1.77 | 2.35 | 2.35 | 4.41 |
| Sulfate Level | 3.80 | 3.80 | 3.64 | 4.17 | 3.36 |
| Ethoxylate:Sulfate (Ratio) | 0.46 | 0.46 | 0.65 | 0.56 | 1.31 |

¹Copolymer of Acrylamide(AM) and TRIQUAT, MW = 1,000,000; CD = 1.6 meq./
gram; AM:TRIQUAT ratio = 95:5
²Copolymer of Acrylamide(AM) and TRIQUAT, MW = 400,000; CD = 1.6 meq./
gram; AM:TRIQUAT ratio = 95:5
³Sodium Laureth Sulfate, supplier: P&G
⁴Sodium Lauryl Sulfate, supplier: P&G
⁵Dimethicone Fluid, Viscasil 330M; 30 micron particle size; supplier: General
Electric Silicones
⁶Tegobetaine F-B, supplier: Goldschmidt Chemicals
⁷Monamid CMA, supplier Goldschmidt Chemical
⁸Ethylene Glycol Distearate, EGDS Pure, supplier Goldschmidt Chemical
⁹Sodium Chloride USP (food grade), supplier Morton.

| | EXAMPLE COMPOSITION | | | | |
|---|---|---|---|---|---|
| Ingredient | XXVIII | XXIX | XXXVII | XXXVIII | XXXIX |
| Water | q.s. | q.s. | q.s. | q.s. | q.s. |
| AM:TRIQUAT Copolymer¹ | 0.25 | 0.25 | — | 0.25 | 0.10 |
| AM:TRIQUAT Copolymer² | — | — | 0.25 | — | — |
| Polyquaternium 10³ | 0.10 | — | — | — | 0.10 |
| Polyquaternium 10⁴ | — | 0.10 | — | — | — |
| Polyquaternium 10⁵ | — | — | 0.10 | — | — |
| Guar Hydroxypropyl Trimonium | — | — | — | 0.10 | — |
| Chloride⁶ | | | | | |
| Sodium Laureth Sulfate (SLE3S)⁷ | 10.00 | 8.00 | 6.00 | 10.00 | 8.00 |
| Sodium Lauryl Sulfate (SLS)⁸ | 4.00 | 6.00 | 10.00 | 6.00 | 10.00 |
| Dimethiconol Microemulsion A⁹ | 1.00 | — | — | — | 0.50 |
| Dimethiconol Microemulsion B¹⁰ | — | 1.00 | 1.00 | 1.00 | — |
| Cocoamdopropyl Betaine¹³ | 2.00 | 4.00 | 4.00 | 2.00 | — |
| PPG-2 Hydroxyethyl | 2.00 | — | — | 1.00 | 2.00 |
| Coco/Isostearamide¹⁴ | | | | | |
| Sodium Chloride¹⁵ | 1.25 | 1.00 | 1.25 | 1.25 | 1.00 |
| Fragrance | 0.70 | 0.70 | 0.70 | 0.70 | 0.70 |
| Preservatives, pH adjusters | Up to | Up to | Up to | Up to 1% | Up to |
| | 1% | 1% | 1% | | 1% |
| Calculated: | | | | | |
| Ethoxylate level | 2.94 | 2.35 | 1.77 | 2.94 | 2.35 |
| Sulfate level | 2.95 | 3.11 | 3.80 | 3.48 | 4.17 |
| Ethoxylate:Sulfate (Ratio) | 1.00 | 0.76 | 0.46 | 0.84 | 0.56 |

¹Copolymer of Acrylamide(AM) and TRIQUAT, MW = 1,000,000; CD = 1.6 meq./
gram; AM:TRIQUAT ratio = 95:5
²Copolymer of Acrylamide(AM) and TRIQUAT, MW = 400,000; CD = 1.6 meq./
gram; AM:TRIQUAT ratio = 95:5
³Polyquaterium 10 polymer with MW = 2.0 MM and charge density = 0.7
⁴UCare Polymer JR30M, MW = 2.0 MM, charge density = 1.32 meq./gram,
supplier Dow Chemicals
⁵UCare Polymer KG30M, MW = 2.0 MM, charge density = 1.96 meq./gram,
supplier Dow Chemicals
⁶Jaguar Excel, supplier: Rhodia.
⁷Sodium Laureth Sulfate, supplier: P&G
⁸Sodium Lauryl Sulfate, supplier: P&G
⁹Dow Corning Silicone Micro-emulsion DC2-1870; Internal Phase Viscosity = 72,000;
30 nm particle size dimethiconol using TEA dodecyl benzene sulfonate
and laureth 23 as primary surfactants
¹⁰Dow Corning DC2-1865 batch#19238-8; Internal Phase Viscosity = 25,000 cps;
25 nm particle size dimethiconol using TEA dodecyl benzene sulfonate and
laureth 23 as primary surfactants
¹¹Tegobetaine F-B, supplier: Goldschmidt Chemicals
¹²Promidium 2, supplier Unichema
¹³Sodium Chloride USP (food grade), supplier Morton.

| | EXAMPLE COMPOSITION | | | | | |
|---|---|---|---|---|---|---|
| Ingredient | XL | XLI | XLII | XLIII | XLIV | XLV |
| Water | q.s. | q.s. | q.s. | q.s. | q.s. | q.s. |
| AM:TRIQUAT | 0.25 | 0.50 | — | 0.50 | 0.1 | 0.25 |
| Copolymer¹ | | | | | | |
| Sodium Laureth Sulfate² | 6.00 | 8.00 | 10.00 | 8.00 | 8.00 | 12.00 |
| Sodium Lauryl³ | 10.00 | 8.00 | 6.00 | — | — | 2.00 |
| Aminosilicone⁴ | 2.00 | — | — | — | — | — |
| Aminosilicone⁵ | — | — | — | — | — | 2.00 |
| Di-PPG-2 Myreth-10 | — | 1.00 | — | — | — | — |
| Adipate⁶ | | | | | | |
| Zinc Pyrithione⁷ | — | — | — | — | — | 1.00 |
| Cocoamdopropyl Betaine⁸ | — | 2.00 | 2.00 | 8.00 | 8.00 | 2.00 |
| Silica⁹ | — | 1.00 | — | — | — | — |
| Silica¹⁰ | — | — | 1.00 | — | — | — |
| Silica¹¹ | — | — | — | 1.00 | — | — |
| Silica¹² | — | — | — | — | 1.00 | — |
| Polymethylsilsesquioxane A¹³ | — | — | 1.00 | — | — | — |
| Polymethylsilsesquioxane B¹⁴ | — | — | — | 1.00 | — | — |
| PPG-2 Hydroxyethyl | 2.00 | 2.00 | — | 2.00 | 2.00 | 2.00 |
| Coco/Isostearamide¹⁵ | | | | | | |
| Ethylene Glycol Distearate¹⁶ | 1.50 | 1.50 | 1.50 | 1.50 | 1.50 | 1.50 |
| Sodium Chloride¹⁷ | 1.50 | 1.50 | 1.50 | 1.50 | 1.50 | 1.50 |
| Fragrance | 0.70 | 0.70 | 0.70 | 0.70 | 0.70 | 0.70 |
| Preservatives, pH adjusters | Up | Up | Up | Up to | Up | Up |
| | to | to | to | 1% | to | to |
| | 1% | 1% | 1% | | 1% | 1% |
| Calculated: | | | | | | |
| Ethoxylate level | 1.77 | 2.35 | 2.94 | 2.35 | 2.35 | 3.53 |
| Sulfate level | 3.80 | 3.64 | 3.48 | 1.51 | 1.51 | 2.79 |
| Ethoxylate:Sulfate (Ratio) | 0.46 | 0.65 | 0.84 | 1.56 | 1.56 | 1.26 |

¹Copolymer of Acrylamide(AM) and TRIQUAT, MW = 400,000; CD = 1.6 meq./
gram; AM:TRIQUAT ratio = 95:5
²Sodium Laureth Sulfate, supplier: P&G
³Sodium Lauryl Sulfate, supplier: P&G
⁴Aminosilicone; supplier General Electric; terminal aminopropyl substitution,
viscosity 350,000, D 1600, M′ = 2, particle size 3 μm
⁵DC 2-8194 Aminosilicone; supplier Dow Corning, particle size ~30 nm
⁶Cromollient SCE, supplier Croda
⁷Zinc Pyrithione, supplier: Arch Chemicals
⁸Tegobetaine F-B, supplier: Goldschmidt Chemical
⁹Sipernat 22LS, supplier: Degussa
¹⁰MSS-500/H, supplier: General Electric Silicones
¹¹MSS-500/N, supplier: General Electric Silicones
¹²Syloid 244FP Silica, supplier: Grace Davison
¹³Tospearl 240, supplier: General Electric Silicones
¹⁴Tospearl 3120, supplier: General Electric Silicones
¹⁵Promidium 2, supplier Unichema
¹⁶Ethylene Glycol Distearate, EGDS Pure, supplier Goldschmidt Chemical
¹⁷Sodium Chloride USP (food grade), supplier Morton.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein are, in relevant part, incorporated herein by reference: the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A personal care composition comprising:
a) a synthetic random copolymer having a net positive charge comprising;
i.) a nonionic monomer unit of the following formula: 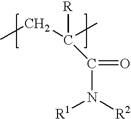
where R is H or C₁₋₄ alkyl; and R¹ and R² are independently selected from the group consisting of H, C₁₋₄ alkyl, CH₂OCH₃, CH₂OCH₂CH(CH₃)₂, and phenyl, or together are C₃₋₆cycloalkyl; and
ii.) a cationic monomer unit with 2 or more positive charges of the following formula: 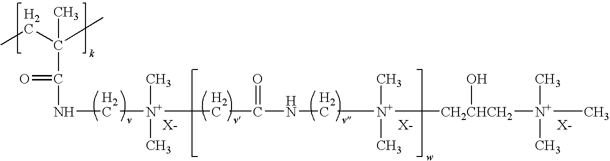
where k=1, each of v, v′, and v″ is independently an integer of from 1 to 6, w is zero or an integer of from 1 to 10, and X⁻ is an anion.
and;
b) an effective amount of a pyrithione or a polyvalent metal salt of a pyrithione;
c) an effective amount of a zinc-containing layered material;
d) a detersive surfactant; and
e) an aqueous carrier.

2. A personal care composition according to claim 1 wherein R, R¹ and R² of said nonionic monomer unit are H and further wherein v=0 and w=1 in said cationic monomer unit.

3. A personal care composition according to claim 1 wherein said synthetic random copolymer is present in an amount from about 0.075% to about 1%, by weight of said personal care composition.

4. A personal care composition according to claim 1, wherein the pyrithione or polyvalent metal salt of pyrithione is zinc pyrithione.

5. A personal care composition according to claim 4 wherein the zinc pyrithione is present from about 0.01% to about 5%.

6. A personal care composition according to claim 1 wherein the zinc-containing layered material is selected from the group consisting of basic zinc carbonate, zinc carbonate hydroxide, hydrozincite, zinc copper carbonate hydroxide, aurichalcite, copper zinc carbonate hydroxide, rosasite, phyllosilicate containing zinc ions, layered double hydroxide, hydroxy double salts and mixtures thereof.

7. A personal care composition according to claim 6 wherein the zinc-containing layered material is selected from the group consisting of zinc carbonate hydroxide, hydrozincite, basic zinc carbonate and mixtures thereof.

8. A personal care composition according to claim 7 wherein the zinc-containing layered material is hydrozincite or basic zinc carbonate.

9. A personal care composition according to claim 8 wherein the zinc-containing layered material is basic zinc carbonate.

10. A personal care composition according to claim 1 wherein said detersive surfactant comprises at least one anionic surfactant having an ethoxylate level and an anion level;
a) wherein said ethoxylate level is from about 1 to about 10, and
b) wherein said anion level is from about 1 to about 10.

11. A personal care composition according to claim 1, wherein said detersive surfactant is selected from the group consisting of sulfates, sulfonates, sulfosuccinates, isethionates, carboxylates, phosphates, phosphonates and mixtures thereof.

12. A personal care composition according to claim 1, wherein said detersive surfactant is selected from the group consisting of amphoteric surfactants, zwitterionic surfactants, cationic surfactants, nonionic surfactants and mixtures thereof.

13. A personal care composition according to claim 12 wherein said amphoteric or zwitterionic surfactant is selected from the group consisting of cocoamidopropyl betaine, cocobetaine, cocoamphoacetate, cocoamphodiacetate, lauroamphoacetate, lauroamphodiacetate, lauramine oxide and mixtures thereof.

14. A personal care composition according to claim 1 wherein lather volume is from about 50 ml to about 500 ml at the 40 second measurement.

15. A personal care composition according to claim 1 wherein said synthetic random copolymer is present in an amount from about 0.01% to about 5% by weight of said personal care composition.

16. A personal care composition according to claim 1 wherein said synthetic random copolymer has a linear charge density from at least about 2 meq/A to about 500 meq/A.

17. A personal care composition according to claim 1 wherein said synthetic random copolymer has a mass charge density from about 0.1 meq/gm to about 6.0 meq/gm.

18. A personal care composition according to claim 1 wherein said synthetic random copolymer has a molecular weight from about 10,000 to about 10,000,000.

19. A personal care composition according to claim 1, further comprising a cationic guar, cationic cellulose polymer, or a cationic modified starch polymer.

20. A personal care composition according to claim 1, further comprising at least one conditioning agent.

21. A personal care composition according to claim 20, wherein said conditioning agent is present in an amount from about 0.01 wt. % to about 10 wt. %.

22. A personal care composition according to claim 21, wherein said conditioning agent is selected from the group consisting of silicone conditioning agents, hydrocarbon oils, polyolefins, fatty alcohols, fatty esters, and mixtures thereof.

23. A personal care composition according to claim 22, wherein said silicone conditioning agent has a particle size of less than or equal to about 50 μm.

24. A personal care composition according to claim 22, wherein said silicone conditioning agent is selected from the group consisting of organo-modified silicones and fluoro-modified silicones.

25. A personal care composition according to claim 24, wherein said organo-modified silicone comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups.

26. A personal care composition according to claim 1, further comprising one or more additional components selected from the group consisting of anti-dandruff actives, particles, opacifying agents, suspending agents, paraffinic hydrocarbons, propellants, and a mono- or divalent salt.
